(19) United States
(12) Patent Application Publication
(10) Pub. No.: US 2008/0103121 A1
(43) Pub. Date: May 1, 2008

(54) CEPHALOSPORIN DERIVATIVE FORMULATION

(76) Inventors: Dilip Jagannath Gole, Plainsboro, NJ (US); Ketan Amin, Randolph, NJ (US); M. llias Jimidar, Turnhout (BE); Hans Vermeersch, Gent (BE); Michael Tran, Cheltenham, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/874,405

(22) Filed: Oct. 18, 2007

Related U.S. Application Data
(60) Provisional application No. 60/855,240, filed on Oct. 30, 2006.

(51) Int. Cl. A61K 31/546; A61P 31/04

(52) U.S. Cl. 514/202

(57) ABSTRACT

The present invention relates to a freeze-dried formulation for cephalosporin derivatives having increased stability and a method for preparing such a formulation using certain excipients for stabilizing the formulation.

### FIELD OF THE INVENTION

The present invention relates to a freeze-dried formulation for a cephalosporin and derivatives thereof having increased stability and a method for preparing such a formulation using certain excipients for stabilizing the formulation.

### BACKGROUND OF THE INVENTION

Active pharmaceutical ingredients (API) are susceptible to degradation in a formulation depending on intrinsic as well as extrinsic factors. The intrinsic factors depend on the ingredients used in the formulation and their interaction with the API in the final form over time depending on various extrinsic factors. Additional intrinsic variables for a freeze-dried formulation include the formulation pH during manufacturing and after reconstitution, the quantity of acidic and alkaline ingredients (excipients) used and their interaction together and with the API. Certain extrinsic factors include the process conditions during manufacturing, environmental conditions during storage or prior to use and length of time after reconstitution. Examples of process conditions include the temperature, pressure and time taken for each step in freezing, sublimation and drying of the formulation. Examples of environmental conditions include temperature, light, oxygen, humidity and length of time under stress conditions, particularly during a stability study.

European Patent EP 1087980 describes a cephalosporin derivative of the present invention in a freeze-dried formulation.

PCT Application WO 06/050631 (Stabilized Freeze-dried Formulation for Cephalosporin Derivatives) describes a freeze-dried formulation for a cephalosporin derivative of the present invention having increased stability, a solution for obtaining and a method for preparing such a formulation, as well as the use of certain compounds for stabilizing cephalosporin derivatives in freeze-dried formulations. The compounds preferably used as stabilizers according to the invention are mannitol, trehalose, and PVP. The advantages and disadvantages of using certain excipients in a freeze-dried formulation are discussed, concluding that the scientific literature on the subject of the effect of excipients on the stabilization of pharmaceutical active ingredients gives contradictory information on their properties and furthermore does not make it possible to obtain some information on the subject of the relationships between the structure of the freeze-dried product and its stability. Likewise, the role of the polyols (such as mannitol) and of the amino acids, alone or in combination, is not described according to a set of generalizable properties, but has been observed with contradictory results according to the API studied and the quantities of excipients used.

Based on the prior art, there remains a need for a freeze-dried formulation for a cephalosporin derivative of the present invention having an increased stability during manufacture, while stored prior to use and post-reconstitution.

### SUMMARY OF THE INVENTION

The present invention is directed to a freeze-dried formulation for a cephalosporin and derivatives thereof.

According to the present invention, it was surprisingly found that the presence and absence of certain ingredients have an unexpected effect on the stability of a freeze-dried cephalosporin derivative formulation.

The present invention will direct the attention of one skilled in the art to those ingredients that markedly improve the stability of the subject formulation during lyophilization, during storage and during the time of post-reconstitution use.

The present invention will also further exclude those ingredients used in previous formulations that have demonstrated an ability to increase the degradation rate of a cephalosporin derivative.

### DETAILED DESCRIPTION OF THE INVENTION

The present invention is directed to a freeze-dried formulation comprising a cephalosporin and derivatives thereof and a buffer system. The instant cephalosporin derivative is an API selected from ceftobiprole or a form thereof.

The term “form” refers to an API present in the formulation of the present invention as a pharmaceutically acceptable salt, stereoisomer, tautomer, crystalline, polymorph, amorphous, solvate, hydrate, ester, prodrug or metabolite thereof.

The API contained in the formulation of the present invention may be a single active ingredient or may be combined with another antimicrobial or antiviral active ingredient of chemical (small molecule), protein (macromolecule) or nonprotein nature. Further, the API may be of natural, semi-synthetic or synthetic origin, including combinations of origins.

The cephalosporin derivative of the present invention is ceftobiprole, an anti-methicillin-resistant staphylococcus aureus (MRSA) injectable cephalosporin, also active against other important Gram-positive and Gram-negative bacteria, of Formula (I): 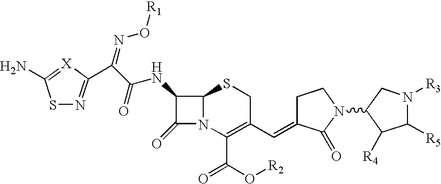
wherein
R₁ is hydrogen, C₁₋₆alkyl, optionally substituted by fluoro or C₃₋₆cycloalkyl;
R₂ is hydrogen or a group selected from —CH₂C(═CHR)—COOR, —CH₂OCOR, —CH(R)OCOR, —CH(R)OCOOR, —CH(OCOR)OCOR, —CH₂COCH₂OCOR and 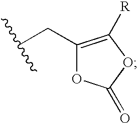
R₃ is hydrogen or a group selected from —CH₂C(═CH₂)—COOR, —COOCH₂C(═CHR)—COOR, —COOCH₂OCOR, —COOCH(R)OCOR, —COOCH(R)OCOOR, —COOCH(OCOR)OCOR, —COOCH₂COCH₂OCOR, and 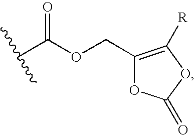
with the proviso that one of R₂ and R₃ is hydrogen and the other of R₂ and R₃ is different from hydrogen;
R is hydrogen or C₁₋₆alkyl;
R₄ is hydrogen or hydroxy;
R₅ is hydrogen or ω-hydroxyalkyl;
X is CH or N;
and a form thereof.

Examples of the compounds of Formula (I) include (6R,7R)-7-[(Z)-2-(amino-[1,2,4]thiadiazol-3-yl)-2-hydroxyimino-acetylamino]-3-[(E)-(3′R, 5′R)-5′-hydroxymethyl-1′-(5-methyl-2-oxo-[1,3]dioxol-4-ylmethyloxycarbonyl-2-oxo-[1,3′]lbipyrrolidinyl-3-ylidenemethyl]-8-oxo-5-thia-1-aza-bicyclo[4.2.0]oct-2-ene-2-carboxylic acid or a form thereof, wherein

R₁, R₂, R₄ and R₅ are all hydrogen;

R₃ is 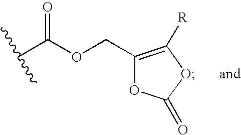
R is methyl.

Examples of the compounds of Formula (I) include the prodrug form of ceftobiprole Formula (Ia) or a form thereof (also referred to as ceftobiprole medocaril) is a carbamate moiety that has a high aqueous solubility, thus making Formula (Ia) suitable for parenteral application. In solution, the degradation rate of the prodrug Formula (Ia) or a form thereof to the ceftobiprole metabolite Formula (Ib) or a form thereof is pH and temperature dependent. The rate of degradation increases as pH and temperature increase. In plasma, cleavage of the prodrug Formula (Ia) to the ceftobiprole metabolite Formula (Ib) (active moiety) occurs rapidly. Formula (Ia):

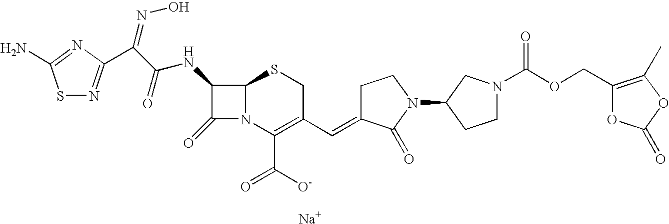

Examples of the compounds of Formula (I) also include the ceftobiprole metabolite Formula (Ib) or a form thereof, the ceftobiprole free acid Formula (Ic) or a form thereof and the ceftobiprole trihydrate hydrochloride salt Formula (Id) or a form thereof:

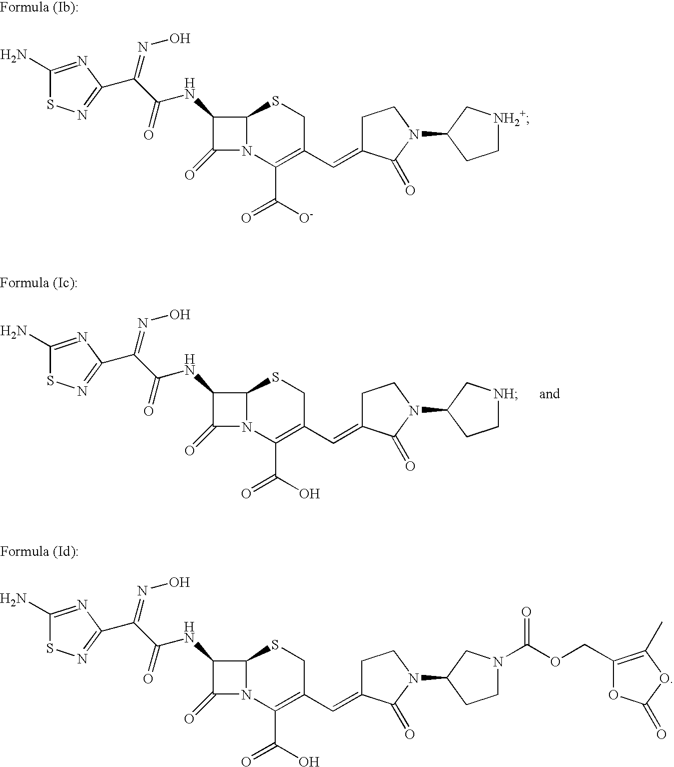

Prior art formulations provide the prodrug form as a sterile lyophilized product which is reconstituted using water for injection or other suitable infusion vehicle.

In one such reconstituted prior art embodiment, the prodrug form (333.3 mg) uses of citric acid (5.25 mg, 10 mM), and sodium hydroxide to adjust the pH to 4.5 and is then reconstituted and diluted for intravenous injection.

In a second reconstituted prior art embodiment, the prodrug form (666.6) uses of citric acid (10.5 mg, 10 mM), and sodium hydroxide to adjust the pH to 4.5 and is then reconstituted and diluted for intravenous injection.

In the prior art embodiments, the prior art formulation comprises the prodrug form, mannitol and a citrate buffer. In the formulation of the present invention, we have discovered that mannitol is not required and its removal results in an improvement to such prior art embodiments.

An example of such a prior art formulation includes a Formulation (I) obtained from water for injection or other suitable infusion vehicle, the prodrug form (333.3 mg), mannitol (15% w/w) and citric acid monohydrate (5.25 mg) and sodium hydroxide to adjust the pH.

Another example of such a prior art formulation includes a Formulation (II) obtained from water for injection or other suitable infusion vehicle, the prodrug form (666.6 mg), mannitol (15% w/w) and citric acid monohydrate (10.5 mg) and sodium hydroxide to adjust the pH. These formulations are similarly then reconstituted and diluted for intravenous injection.

In the prior art embodiments, the prior art formulation required a reconstitution diluent for the prior art formulations is obtained from water for injection, 10 mg/mL of dextrose (glucose), 8.4 mg/mL of citric acid monohydrate (40 mM) and sodium hydroxide to adjust pH to 5.0. In the formulation of the present invention, we have discovered that use of a buffer system in the formulation mixture added prior to freeze-drying results in an improvement to such prior art embodiments.

Due to the complexity of the Formula (I) chemical structure and features, the prodrug Formula (Ia) exhibits limited stability, the robustness of which is further difficult to predict based on compatibility with the excipients used in the formulation, particularly in biocompatible formulations and the like. These characteristics challenge the ordinary skills and conventional methodologies in this technology, particularly when it comes to the preparation of lyophilized prodrug formulations that are to be readily used for medical purposes. Such uses rely on formulations with characteristics such as biocompatibility, stability under ambient conditions, or under conditions that are as near to ambient conditions as possible, with a shelf life that is as long as possible, having ease of reconstitution and forming reconstituted solutions that are as stable under ambient, or near ambient conditions, for as long as possible. This goal is achieved in the present invention by balancing formulation pH, buffer concentration and buffer capacity in the formulation mixture prior to freeze-drying. There remains a need therefore, for a formulation and reconstitution methodology for preparing such formulations that provide the desirable features and characteristics such as those referred to above.

In the practice of the prior art, Formulation (I) is manufactured by freeze-drying 2.5 mL of solution containing the ceftobiprole prodrug, mannitol, citric acid buffer and water in a vial. A prior art Formulation (II) is similarly manufactured by freeze-drying 5 mL of the solution.

The prior art freeze-drying process for the prior art formulation involves lyophilizing the aqueous solution or suspension containing the API and excipients in the market container by freezing the solution or suspension, then reducing the pressure in the freeze chamber for a period of primary drying. The fill volume of Formulation (I) (2.5 mL) and Formulation (II) (5 mL) must be placed in the market container prior to freezing. The initial drying step removes water vapor from the frozen material by sublimation and gives a semi-dried mass. The temperature is then increased for a period of secondary drying to remove residual water from the semi-dried mass. The market container is then sealed. The prior art lyophilized formulation is then stored and later reconstituted with a reconstitution solvent, wherein the solvent has a high buffer concentration (40 mM) or buffer capacity and sodium hydroxide to provide a post-reconstitution pH 5.0.

As described above, the prior art Formulation (I) and Formulation (II) have several disadvantages. PCT Application WO 06/050631 describes the use of stabilizers such as amino acids, carbohydrates, polyhydric alcohols and polyvinyl pyrrolidinone (PVP), including mannitol, trehalose, and PVP. An understanding of the effect of excipients on the stabilization of an API is often not an exact science but more of an art. Often, one skilled in the art must conduct elaborate design studies to elucidate the interaction between various combinations of excipients and the API because the properties of the excipients interacting in combination does not make it possible to understand the effect on the stability of the final freeze-dried product. Accordingly, the interaction of a polyhydric alcohol excipient, particularly mannitol, in such combinations may not act according to a set of generalized properties, but has been observed with contradictory results according to the API studied and the quantity of excipient used. We have discovered that the use of mannitol in the lyophilized formulation results in an adduct of Formula (Ie) formed with the prodrug Formula (Ia) after reconstitution, thus inactivating the prodrug form and limiting the shelf life of the product. Accordingly, the use of mannitol is contraindicated. Formula (Ie):

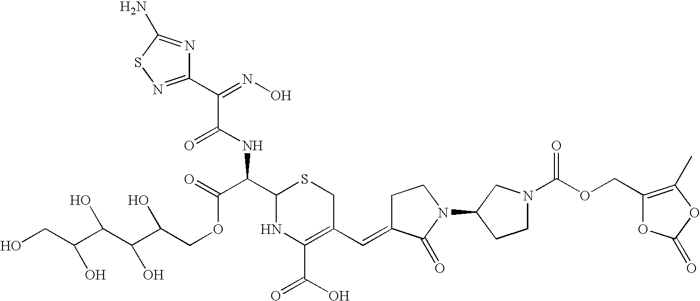

After reconstitution, the prior art Formulation (I) and Formulation (II) (at pH 4.5 using 10 mM citrate buffer during manufacture) forms an opalescent or turbid solution when commercial vehicles such as water for injection or 5% dextrose injection are used for reconstitution. Such turbidity is an undesirable property and demands the use of a special reconstitution solvent having a high buffer capacity and sodium hydroxide that provides a post-reconstitution pH 5.0 and results in a clear reconstituted solution.

The formulation of the present invention provides a buffer system added to the formulation mixture prior to lyophilization that results in a higher post-reconstitution pH range and a higher buffer concentration, thus enhancing the appearance and stability of the reconstituted and infusion solutions, thereby eliminating the need for the prior art special reconstitution solvent.

Furthermore, the prior art reconstituted Formulation (I) and Formulation (II) have an inherent lower buffer capacity, thus making the formulations sensitive to pH drifts in commercial infusion vehicles. Such sensitivity is also an undesirable property from a stability standpoint and has heretofore been overcome by using said high buffer capacity reconstitution solvent. However, even with the use of the high buffer capacity reconstitution solvent, the pH sensitivity of the prior art reconstituted Formulation (I) and Formulation (II) significantly reduced usage life and allowed for only a short handling time of the infusion solution.

The prior art freeze-drying process, whereby a liquid volume of Formulation (I) (2.5 mL) and Formulation (II) (5 mL) is placed in the market container prior to freezing, may be made more advantageous and efficient by using a bulk lyophilization process followed by aseptic filling of dried powder into the market container. The production of a sterile formulation by such a bulk lyophilization process would significantly increase manufacturing output.

The formulation of the present invention comprises ingredients which improve stability of the final form during manufacture, while stored prior to use and post-reconstitution.

The present invention provides a composition in the form of a lyophilized formulation, comprising a compound of Formula (Ia) and a buffer system.

The present invention provides a composition in the form of a lyophilized formulation, comprising a compound of Formula (Id) as a trihydrate hydrochloride salt and a buffer system.

The present invention provides a composition in the form of a bulk lyophilized formulation, comprising a compound of Formula (Ia) and a buffer system.

The present invention provides a composition in the form of a bulk lyophilized formulation, comprising a compound of Formula (Id) as a trihydrate hydrochloride salt and a buffer system.

The term “formulation” or “composition” refers to a product containing one or more compounds of Formula (I), Formula (Ia), Formula (Ib), Formula (Ic), Formula (Id) or a form thereof (such as a product comprising the specified ingredients in the specified amounts, as well as any product which results, directly or indirectly, from such combinations of the specified ingredients in the specified amounts). The terms composition and formulation are used interchangeably, whereby both terms are intended to have a similar meaning and both of which, in addition to the foregoing definition, are intended to take on the ordinary meaning given to them by one skilled in the art.

The formulation of the present invention may further comprise one or more additional optional ingredients selected from a bulking agent, a surfactant, a preservative, an antioxidant, a chelating agent or an optional cosolvent system.

While certain additional ingredients may lend a stabilizing effect to the formulation, the additional optional ingredients included above are well-known pharmaceutically acceptable excipients often used in freeze-dried forms. Further customary additives known to one skilled in the preparation of pharmaceutical formulations such as flavouring agents or dyes may be added as well.

The buffer system of present invention comprises one or more biocompatible ingredients selected from an acid, a base and a salt.

Embodiments of the present invention include an acid or a base which is mono-acidic, mono-basic, poly-basic or poly-acidic. Biocompatible buffer systems that permit the control of pH at a desired value provide additional embodiments of this invention. It is understood by one skilled in the art that the acids and bases composing said buffer system may also be introduced alone, including hydrates, as well as any combinations thereof. The mono-, di- and tricarboxy citric acids are included and will exist as either mono-, di- and tricarboxy depending on the formulation's final pH. The choice of ingredients for use in the buffer system of the present invention are within the knowledge of one skilled in the art to result in a buffer system which is compatible with the API and a salt thereof.

Embodiments of the present invention include a buffer system comprising an acid, a base and a salt selected from acetate, acetic acid, arginine, ascorbate, ascorbic acid, bicarbonate, carbonate, carbonic acid, citrate, citric acid, glutamate, glutamic acid, glycine, histidine, hydrochloric acid, hydrogen carbonate, lactate, lactic acid, maleate, maleic acid, phosphate, phosphoric acid, potassium dihydrogen phosphate, potassium hydroxide, sodium dihydrogen phosphate, sodium hydroxide, succinate, succinic acid, tartrate, tartaric acid, tri(hydroxymethyl)aminomethane (TRIS) and combinations thereof.

Embodiments of the present invention further include a buffer system comprising an acid, a base and a salt selected from acetate, acetic acid, arginine, ascorbate, ascorbic acid, bicarbonate, citrate, citric acid, glutamate, glutamic acid, glycine, histidine, hydrochloric acid, lactate, lactic acid, phosphate, phosphoric acid, potassium dihydrogen phosphate, potassium hydroxide, sodium dihydrogen phosphate, succinate, succinic acid, tartrate, tartaric acid and combinations thereof.

Embodiments of the present invention also include a buffer system comprising an acid or salt thereof selected from citrate, citric acid, glutamic acid, hydrochloric acid, phosphate, phosphoric acid and combinations thereof.

Embodiments of the present invention include buffers system comprising a combination of an acid, a base and a salt selected from potassium dihydrogen phosphate, phosphate/citrate, sodium dihydrogen phosphate, tartrate/citrate and the like.

An example of the present invention is a buffer system comprising an acid, a base and a salt, wherein the acid is selected from citric acid, glutamic acid, hydrochloric acid, phosphoric acid and combinations thereof; and, wherein the base is selected from potassium hydroxide or sodium hydroxide and combinations thereof.

Another example of the present invention is a buffer system comprising an acid, a base and a salt, wherein the acid is citric acid; and, wherein the base is sodium hydroxide.

The concentration (mM) of the buffer system in embodiments of this invention is determined according to the solubility and compatibility of the acid, base and salt used therein. For a freeze-dried formulation, another factor for determining the concentration depends on the ability of the buffer system to be freeze-dried.

Embodiments of the present invention include a buffer system concentration in a range of about 1 mM, or of about 10 mM, or of about 25 mM, or of from about 10 mM to about 25 mM, or of from about 10 mM to about 35 mM, or of from about 10 mM to about 40 mM, or of from about 10 mM to about 50 mM, or of from about 10 mM to about 100 mM, or of from about 25 mM to about 35 mM, or of from about 25 mM to about 40 mM, or of from about 25 mM to about 50 mM, or of from about 25 mM to about 100 mM, or of from about 25 mM to about 200 mM, or of from about 50 mM to about 200 mM.

Examples of the present invention include a buffer system concentration in a range of about 25 mM, or of from about 10 mM to about 50 mM, or of from about 25 mM to about 50 mM, or of from about 25 mM to about 200 mM, or of from about 50 mM to about 200 mM.

Examples of the present invention also include a buffer system concentration in a range of about 25 mM, or of from about 10 mM to about 50 mM.

Examples of the present invention further include a buffer system concentration in a range of about 25 mM.

Embodiments of the present invention include bulking agents such as, without limitation, cellobiose, cyclodextrin, gelatin, gentiobiose, isomaltose, isosaccharose, isotrehalose, lactose, maltodextrins, maltose, melibiose, PVP, sorbose, sucralose, sucrose or trehalose or turanose.

The ratio of the bulking agent to the API in embodiments of this invention is determined according to the solubility of the bulking agent. For a freeze-dried formulation, another factor for determining the ratio depends on ability of the bulking agent to be freeze-dried.

Embodiments of the present invention include a weight/weight (w/w) ratio of bulking agent:API (bulking agent to API) in a range of about 0:1, or of about 1:5, or of about 1:10, or of about 3:100, or of from about 1:10 to about 0:1, or of from about 1:10 to about 1:100, or of from about 1:100 to about 5:100, or of from about 1:200 to about 1:800, or of from about 1:250 to about 1:600, or of from about 1:100 to about 1:1500.

Embodiments of the present invention further include a (w/w) ratio of bulking agent:API in a range of from about 1:100 to about 5:100, or of from about 1:200 to about 1:800, or of from about 1:250 to about 1:600, or of about 3:100.

Embodiments of the present invention also include a (w/w) ratio of bulking agent:API in a range of about 3:100.

Embodiments of the present invention include surfactants such as, without limitation, phospholipids (such as lecithin), polysorbates, poloxamers (such as polyoxyethylene 20 sorbitan monooleate or polyoxyl 40 stearate), tyloxapol, polyoxyethylene-polyoxypropylene copolymers (such as a Pluronic surfactant), polyoxyethylene esters of 12-hydroxystearic acid (such as a Solutol surfactant), ethoxylates of cholesterol (such as diacyl glycerol or dialkyl glycerol), bile salts (such as sodium cholate or sodium deoxycholate), sucrose esters (such as sucrose monolaurate or sucrose monooleate) or polyvinyl alcohol (PVA) and the like.

Embodiments of the present invention include cephalosporins and derivatives thereof and the like in the form of pharmaceutically acceptable salts. For use in medicines, the “pharmaceutically acceptable salts” of a cephalosporin derivative of this invention refers to non-toxic acidic/anionic or basic/cationic salt forms.

Suitable salt forms include acid addition salts which may, for example, be formed by mixing a solution of the cephalosporin derivative according to the invention with a solution of an acid such as acetic acid, adipic acid, benzoic acid, carbonic acid, citric acid, fumaric acid, glycolic acid, hydrochloric acid, maleic acid, malonic acid, phosphoric acid, saccharinic acid, succinic acid, sulphuric acid, tartaric acid, trifluoroacetic acid and the like.

Furthermore, when a cephalosporin derivative of the present invention is bound with an acidic moiety, suitable salts thereof may include alkali metal salts, e.g. sodium or potassium salts; alkaline earth metal salts, e.g. calcium or magnesium salts; and salts formed with suitable organic ligands, e.g. quaternary ammonium salts.

Thus, representative salts and alkali metal or alkaline earth metal salts thereof, include the following: acetate, adipate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, glyconate, hydrabamine, hydrobromine, hydrochloride, iodide, isothionate, lactate, malate, maleate, malonate, mandelate, mesylate, nitrate, oleate, pamoate, palmitate, phosphate/diphosphate, saccharinate, salicylate, stearate, sulfate, succinate, tartrate, tosylate, trichloroacetate, trifluoroacetate and the like.

Embodiments of the present invention include salts such as, without limitation, acetate, bicarbonate, chloride, glutamate, hydrochloride or sodium; alkali metal sodium salts such as ededate (tetrasodium EDTA), docusate (sodium 1,4-bis(2-ethylhexyl)sulphosuccinate), potassium or dipotassium carbonate; or alkaline earth metal salts such as magnesium stearate and, for each of the foregoing, a hydrate thereof.

Embodiments of the present invention include preservatives such as, without limitation, methyl and propyl para-hydroxybenzoate, benzethonium chloride, sodium mercurothiolate, phenylmercuric nitrate, benzyl alcohol, phenol and metacresol.

Embodiments of the present invention include cosolvent systems such as, without limitation, alcohols (such as methanol, ethanol, propanol, t-butanol), glycerin, polyethylene glycol, propylene glycol, vegetable oils and the like.

Embodiments of the present invention include cephalosporins and derivatives thereof and the like with solubility enhancing properties through self solubilization mechanisms.

The formulations according to the present invention may be either reconstituted in liquid form by addition of an adequate solvent or reconstitution solution for its administration via the parenteral, intra-muscular or oral route, or directly administered via the oral route, to a subject. In addition, the liquid or dry formulation may be administered by inhalation.

The cephalosporin derivatives of the present invention include a form thereof and, as such, include a drug as well as a prodrug form thereof.

An example of the present invention includes the use of a buffer system for solubilizing a cephalosporin derivative prior to freezing, sublimating and drying, thereby buffering the formulation's post-reconstitution pH to prevent precipitation and degradation of the cephalosporin derivative.

An example of the present invention also includes the use of a ceftobiprole prodrug of Formula (Ia) “seeded” with a ceftobiprole metabolite of Formula (Ib) to maintain the solubility of the prodrug during IV dosing. Accordingly, the formulation of the present invention may comprise one or more compounds of Formula (I), Formula (Ia), Formula (Ib), Formula (Ic), Formula (Id) and mixtures thereof.

An example of the present invention may comprise a compound of Formula (Ia), a compound of Formula (Ib) and mixtures thereof.

The ceftobiprole metabolite of Formula (Ib) is an inherent degradation product of the solvated prodrug of Formula (Ia), the metabolite is also the in vivo metabolite active present in a subject after dosing. The presence of “seeded” metabolite modulates the degradation of the solvated prodrug by maintaining the solubility of the prodrug after the lyophilized formulation is reconstituted. As a result, the present invention enhances the solubility of the ceftobiprole prodrug and enables an increase in the time for infusion before the prodrug significantly degrades.

The formulation of the present invention “seeded” with the ceftobiprole metabolite of Formula (Ib) in a buffer system provides a formulation that demonstrates a post-reconstitution stability of up to 24-30 hours at 25° C. and may demonstrate a post-reconstitution stability of at least 48 hours at 5° C.

Furthermore, the formulation of the present invention eliminates ingredients, which demonstrably degrade stability of the final form. More specifically, the bulking agent mannitol has been found to particularly increase the degradation rate of a ceftobiprole API during manufacture, storage and use.

The present invention also solves the problems of previous freeze-dried cephalosporin derivative formulations that required a special diluent for reconstitution by providing a formulation that merely requires water for injection. The special diluent of previous formulations was a combination of a buffer, an alkaline solution and dextrose that adjusted the post reconstitution pH to slow the degradation rate of a ceftobiprole API.

Accordingly, the present invention is directed to a freeze-dried cephalosporin derivative formulation comprising one or more cephalosporin derivatives and a buffer system. The one or more cephalosporin derivatives of the present invention are selected from ceftobiprole or a form thereof.

Embodiments of the present invention include a cephalosporin derivative selected from a ceftobiprole prodrug and a metabolite thereof.

In reference to a cephalosporin derivative of the present invention, such may exist as, without limitation, a pharmaceutically acceptable salt, stereoisomer, tautomer, crystalline, polymorph, amorphous, solvate, hydrate, ester, prodrug or metabolite form. The present invention encompasses all such cephalosporin derivative forms and mixtures thereof.

The term “isolated form” means, in reference to a cephalosporin derivative of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such cephalosporin derivative forms and mixtures thereof.

The present invention includes a cephalosporin derivative of various isomers and mixtures thereof. The term “isomer” refers to cephalosporin derivatives that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “stereoisomer” refers to isomers that have the same molecular formula and the same sequence of covalently bonded atoms but a different spatial orientation.

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of the isolated species rotates the plane of polarized light in the opposite direction such that the mixture is devoid of optical activity.

The term “enantiomer” means a stereoisomer that is not nonsuperimposable with its mirror image. The term “diastereomer” means stereoisomers that are not enantiomers.

The term “chiral molecule” means a molecule that has at least one pair of enantiomers. This is in contrast to achiral molecules, which can be superimposed on their mirror images.

The two distinct mirror image versions of the chiral molecule are also known as levo (left-handed), abbreviated L, or dextro (right-handed), abbreviated D, depending on which way they rotate polarized light. The symbols “R” and “S” represent the configuration of groups around a stereogenic carbon atom(s).

An example of an enantiomerically enriched form isolated from a racemic mixture includes a dextrorotatory enantiomer, wherein the mixture is substantially free of the levorotatory isomer. In this context, substantially free means the levorotatory isomer may, in a range, comprise less than 25% of the mixture, less than 10%, less than 5%, less than 2% or less than 1% of the mixture according to the formula:

$$\% levorotatory = \frac{( mass levorotatory )}{( mass dextrorotatory ) + ( mass levorotatory )} \times 100$$

Similarly, an example of an enantiomerically enriched form isolated from a racemic mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrorotatory = \frac{( mass dextrorotatory )}{( mass dextrototatory ) + ( mass levorotatory ) } \times 100$$

The term “geometric isomer” means isomers that differ in the orientation of substituent atoms in relationship to a carbon-carbon double bond, to a cycloalkyl ring, or to a bridged bicyclic system. Substituent atoms (other than hydrogen) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

Substituent atoms (other than hydrogen) attached to a ring system may be in a cis or trans configuration. In the “cis” configuration, the substituents are on the same side in relationship to the plane of the ring; in the “trans” configuration, the substituents are on opposite sides in relationship to the plane of the ring. Atom configurations of a cephalosporin derivative having a mixture of “cis” and “trans” species are designated “cis/trans”.

The isomeric descriptors (“R,” “S,” “E,” and “Z”) indicate atom configurations and are used as defined in the literature.

The cephalosporin derivatives of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, cephalosporin derivatives of the present invention may have one or more polymorph or amorphous crystalline forms and, as such, are intended to be included in the scope of the invention. In addition, said cephalosporin derivatives may form solvates with water (i.e., hydrates) or common organic solvents (e.g., organic esters such as ethanolate and the like) and, as such, are also encompassed within the scope of this invention.

The term “stable” or “a stable prodrug formulation,” refers to a formulation that satisfies the desired stability characteristics as described herein and equivalents thereof that are not possessed by conventional formulations and that are not achieved when the formulation is prepared by conventional manufacturing methodologies.

An embodiment of the present invention is a pharmaceutically acceptable composition comprising a compound of Formula (I) and a buffer system.

An embodiment of the present invention is a pharmaceutically acceptable composition comprising a compound of Formula (Ia) and a buffer system.

An embodiment of the present invention is a pharmaceutically acceptable composition comprising a compound of Formula (Ib) and a buffer system.

An embodiment of the present invention is a pharmaceutically acceptable composition comprising a compound of Formula (Ic) and a buffer system.

An embodiment of the present invention is a pharmaceutically acceptable composition comprising a compound of Formula (Id) and a buffer system.

The present invention is further directed to a method for ameliorating, treating or preventing a chronic or acute disease mediated by anti-methicillin-resistant staphylococcus aureus, a Gram-positive bacteria or a Gram-negative bacteria in a subject in need thereof comprising administering to the subject an effective amount of a reconstituted lyophilized formulation of the present invention.

The present invention is also directed to a use of one or more compounds of Formula (I), Formula (Ia), Formula (Ib), Formula (Ic), Formula (Id) or a form thereof and a buffer system in the manufacture of a medicament for ameliorating, treating or preventing a chronic or acute disease mediated by anti-methicillin-resistant staphylococcus aureus, a Gram-positive bacteria or a Gram-negative bacteria.

The term “reconstituted lyophilized formulation” refers to a lyophilized formulation containing an effective amount of one or more compounds of Formula (I), Formula (Ia), Formula (Ib), Formula (Ic), Formula (Id) or a form thereof and a buffer system.

The term “administering” with respect to the methods of the present invention, refers to a means for treating, ameliorating or preventing a disease as described herein with a reconstituted lyophilized formulation.

Such methods include administering a reconstituted lyophilized formulation at different times during the course of a therapy or concurrently in a combination form. Such methods further include administering a reconstituted lyophilized formulation with one or more agents at different times during the course of a therapy or concurrently in a combination form.

The term “prodrug” refers to a metabolic precursor of a compound of Formula (Ia). In general, a prodrug is a functional derivative of a compound which may be inactive when administered to a subject but is readily convertible in vivo into an active metabolite compound.

The term “active metabolite” refers to a metabolic product of a compound of Formula (I) that is effective for ameliorating, treating or preventing a chronic or acute disease mediated by anti-methicillin-resistant staphylococcus aureus, a Gram-positive bacteria or a Gram-negative bacteria.

The term “subject” as used herein, refers to an animal, a mammal, or a human, who has been the object of treatment, observation or experiment and is at risk of (or susceptible to) developing a chronic or acute disease or having a chronic or acute disease mediated by anti-methicillin-resistant staphylococcus aureus, a Gram-positive bacteria or a Gram-negative bacteria. The term “observation or experiment” includes trials with laboratory tissues, including but not limited to clinical trials, analytical trials, and modelling assays.

The term “effective amount” refers to that amount of active compound or pharmaceutical agent that elicits the biological or medicinal response in a patient's tissue system, animal or human, that is being sought by a researcher, veterinarian, medical doctor, or other clinician, which includes preventing, treating or ameliorating the symptoms of the chronic or acute disease.

The effective amount of a reconstituted lyophilized formulation exemplified in such a method is from about 250 mg to about 500 mg.

The term “medicament” refers to one or more compounds of Formula (I), Formula (Ia), Formula (Ib), Formula (Ic), Formula (Id) or a form thereof used in a product for use in preventing, treating or ameliorating a chronic or acute disease.

A formulation, composition or medicament of the present invention is “pharmaceutically acceptable” when the molecular entities and components used therein are of sufficient purity and quality such that, when appropriately administered to a subject, the formulation, composition or medicament does not produce an adverse, allergic or other untoward reaction. Since both human use (clinical and over-the-counter) and veterinary use are equally included within the scope of the present invention, a pharmaceutically acceptable formulation, composition or medicament for either human or veterinary use.

The term “combination therapy” refers to the use of a formulation, composition or medicament of the present invention in combination with one or more therapeutic agents for preventing, treating or ameliorating a chronic or acute disease and advantageously may facilitate the use of a reduced effective dose of the instant formulation, composition or medicament and/or the therapeutic agent than would be recommended for preventing, treating or ameliorating a chronic or acute disease Therefore, it is contemplated that the formulation, composition or medicament of this invention can be used before, during or after treatment with a particular therapeutic agent.

The term “therapeutic agent” refers to antibacterial agents used for ameliorating, treating or preventing a chronic or acute disease mediated by anti-methicillin-resistant staphylococcus aureus, a Gram-positive bacteria or a Gram-negative bacteria.

The term “ameliorating, treating or preventing” refers, without limitation, to facilitating the eradication of or inhibiting the progression of a chronic or acute disease mediated by anti-methicillin-resistant staphylococcus aureus, a Gram-positive bacteria or a Gram-negative bacteria.

The present invention is directed to a pharmaceutically acceptable composition comprising a compound of Formula (Ia) and a buffer system.

The present invention is also directed to a freeze-dried cephalosporin derivative formulation comprising one or more cephalosporin derivatives and a buffer system, wherein the cephalosporin derivative is a compound of Formula (Ia) and an optionally present compound of Formula (Ic). The amount of the compound of Formula (Ia) and an optionally present compound of Formula (Ic) contained in the market container is selected from 333.3 mg (250 mg dose) or 666.6 mg (500 mg dose).

The freeze-dried formulation is provided in a market container, usually a vial for intravenous injection. The present invention is not limited by specific container forms or designs, though, as long as the container is acceptable for its intended use and meets the standards therefore. Embodiments of this invention are provided with a freeze-dried formulation contained in vials, preferably tubing vials.

The lyophilized formulations of the present invention can be reconstituted with a vehicle and optionally further diluted to give a composition in the form of a solution ready for intravenous injection. The actual amounts of reconstitution vehicle used are not limiting features of embodiments of the invention. By way of illustration, without limitation, embodiments of a reconstitution vehicle for the lyophilized formulation of the present invention include water for injection (WFI), deionized water, demineralized water and the like. The volume of water is in a range of about 10 ml, or of from about 1 ml to about 20 ml, or of from about 1 ml to about 5 ml, or of from about 5 ml to about 10 ml.

Embodiments of a reconstituted freeze-dried formulation of the present invention provide a concentration of the compound of Formula (Ia) and an optionally present compound of Formula (Ic) in a range of about 13.3 mg/ml, or of about 66.7 mg/ml, or of about 133.3 mg/ml, or of about 150.0 mg/ml, or of from about 13.3 mg/ml to about 199.5 mg/ml.

An example of the reconstituted freeze-dried formulation of the present invention provides a concentration of the compound of Formula (Ia) and an optionally present compound of Formula (Ic) in a range of about 66.7 mg/ml.

Reconstituted embodiments of the present invention may optionally be further diluted if so desired, without such dilution being a limitation of the present invention. This optional dilution is preferably carried out with an aqueous system, which is usually 5% dextrose (glucose) or 0.9% sodium chloride or lactated Ringer's. The reconstituted solution may optionally be further diluted depending on the concentration of the API in the reconstituted solution and the desired final concentration of the formulation.

An embodiment of the lyophilization process of the present invention includes freeze-drying the instant formulation in the form of a bulk solution. Embodiments of the buffer system in the bulk solution modulate the bulk solution pH in a range of about pH 4.5 to about pH 5.6. After the lyophilzation process is complete, the dried powder is added by weight to the market container.

Compared to the prior art process wherein the bulk solution is added to the market container prior to being freeze-dried, the lyophilization process of the present invention allows the cephalosporin derivative concentration in the solution to be increased and reduces the fill volume in the market container. The lyophilization process of the present invention allows for the manufacturing of embodiments of bulk solution with an API concentration that is higher than that obtained according to the prior art.

### EXAMPLE 1

### Compositions

Compositions with various buffers were prepared for lyophilization by liquid fill in vials or for bulk lyophilization and powder fill in vials.

The reference formulation contained the compound of Formula (Ia) (666.6 mg), mannitol (Approximately 15% w/w of dry cake weight), citric acid (10 mM), sodium hydroxide solution (q.s. to pH 4.5) and WFI (q.s. to 5 ml).

A test Formula (1) contained the compound of Formula (Ia) (666.6 mg), citric acid (25 mM), sodium hydroxide solution (q.s. to pH 4.8) and WFI (q.s. to 5 ml).

A test Formula (2) contained the compound of Formula (Ia) (666.6 mg), citric acid (10-50 mM), sodium hydroxide or potassium hydroxide solution (q.s. to pH 4.8) and WFI (q.s. to 5 ml).

A test Formula (3) contained the compound of Formula (Ia) (666.6 mg), potassium dihydrogen phosphate (10-200 mM), citric acid (10-50 mM), sodium hydroxide or potassium hydroxide solution (q.s. to pH 4.8) and WFI (q.s. to 5 ml).

A test Formula (4) contained the compound of Formula (Ia) (666.6 mg), sodium dihydrogen phosphate (10-200 mM), citric acid (10-50 mM), sodium hydroxide or potassium hydroxide solution (q.s. to pH 4.8), phosphoric acid (q.s. to pH 4.8) and WFI (q.s. to 5 ml).

A test Formula (5) contained the compound of Formula (Ia) (666.6 mg), histidine (10-50 mM), phosphoric acid (q.s. to pH 4.8), hydrochloric acid (q.s. to pH 4.8) and WFI (q.s. to 5 ml).

A test Formula (6) contained the compound of Formula (Ia) (666.6 mg), glutamic acid (10-50 mM), sodium hydroxide or potassium hydroxide solution (q.s. to pH 4.8) and WFI (q.s. to 5 ml).

A test Formula (7) contained the compound of Formula (Ia) (666.6 mg), arginine (10-50 mM), phosphoric acid (q.s. to pH 4.8) and WFI (q.s. to 5 ml).

A test Formula (8) contained the compound of Formula (Ia) (666.6 mg), glycine (10-50 mM), phosphoric acid (q.s. to pH 4.8), hydrochloric acid (q.s. to pH 4.8) and WFI (q.s. to 5 ml).

A test Formula (9) contained the compound of Formula (Ia) (666.6 mg), sucrose (1-10%), citric acid (10-50 mM), sodium hydroxide or potassium hydroxide solution (q.s. to pH 4.8) and WFI (q.s. to 5 ml).

A test Formula (10) contained the compound of Formula (Ia) (666.6 mg), lactose (1-10%), citric acid (10-50 mM), sodium hydroxide or potassium hydroxide solution (q.s. to pH 4.8) and WFI (q.s. to 5 ml).

A test Formula (11) contained the compound of Formula (Ia) (666.6 mg), cyclodextrin (1-10%), citric acid (10-50 mM), sodium hydroxide or potassium hydroxide solution (q.s. to pH 4.8) and WFI (q.s. to 5 ml).

A test Formula (12) may contain the compound of Formula (Ia) (666.6 mg), trehalose (1-10%), citric acid (10-50 mM), sodium hydroxide or potassium hydroxide solution (q.s. to pH 4.8) and WFI (q.s. to 5 ml).

A test Formula (13) may contain the compound of Formula (Ia) (666.6 mg), sucralose (1-10%), citric acid (10-50 mM), sodium hydroxide or potassium hydroxide solution (q.s. to pH 4.8) and WFI (q.s. to 5 ml).

A test Formula (14) contained the compound of Formula (Ia) (666.6 mg), anionic and non-ionic surfactants (e.g. sodium lauryl sulphate or polysorbate 80) (1-10%), citric acid (10-50 mM), sodium hydroxide or potassium hydroxide solution (q.s. to pH 4.8) and WFI (q.s. to 5 ml).

A test Formula (15) contained the compound of Formula (Ia) (666.6 mg), gelatin (1-5%), citric acid (10-50 mM), sodium hydroxide or potassium hydroxide solution (q.s. to pH 4.8) and WFI (q.s. to 5 ml).

A test Formula (16) contained the compound of Formula (Ia) (666.6 mg), anionic and non-ionic surfactants (e.g. sodium lauryl sulphate or polysorbate 80) (1-10%), chelating agent (0.1-1%), citric acid (10-50 mM), sodium hydroxide or potassium hydroxide solution (q.s. to pH 4.8) and WFI (q.s. to 5 ml).

The lyophilization process of the present invention was performed by filling the bulk solution into vials, reducing the temperature of the solution in the vials to less than 10° C. over a period of about 4 hours, followed by primary drying over a period of about 38 hours, then secondary drying over a period of about 10 hours. After the vial contents was lyophilized, the vials were sealed and stored at −20° C. in a refrigerated storage area. Stability samples were stored at a temperature of 5±3° C. The reference and test formulations were stable at 5° C.

Results

After reconstitution in WFI, the reference formulation formed a turbid solution. After reconstitution in WFI, test Formula (1) was clear (turbidity less than 16 NTU).

Test Formula (1) Stability Results

The stability results for test Formula (1) under various stability conditions are shown in Table 1.

TABLE 1

| | | | 25° C./60% | 25° C./60% | 40° C./75% | 40° C./75% |
|---|---|---|---|---|---|---|
| | Initial | 5° C. 3 Mo | 1 Mo | 3 Mo | 14 days | 1 Mo |
| Appearance | Comply | Comply | Comply | Comply | Comply | Comply |
| Recon. pH | 4.8 | 4.9 | 4.8 | 4.8 | 4.8 | 4.7 |
| Turbidity | 2 | 2 | 2 | 2 | 3 | 2 |
| (<16 NTU) | | | | | | |
| Particulate | 790 | 320 | 890 | 442 | 648 | 2362 |
| Matter | | | | | | |
| (25 μM ≦ 6000) | | | | | | |
| Particulate | 362 | 4 | 38 | 16 | 38 | 146 |
| Matter | | | | | | |
| (10 μM ≦ 6000) | | | | | | |
| Water (% < 3.0) | 2.6 | 0.7 | 0.7 | 0.8 | 0.6 | 0.9 |
| Formula (Ia) | 93.0 | 92.4 | 90.9 | 89.1 | 87.5 | 86.6 |
| % Area | | | | | | |
| Formula (Ic) | 1.2 | 1.2 | 1.5 | 1.7 | 1.8 | 2.0 |
| (% Area ≦ 3.0) | | | | | | |
| Adduct | 1.1 | 1.1 | 1.5 | 2.2 | 2.7 | 3.0 |
| (% Area ≦ 5.0) | | | | | | |
| TRS | 5.3 | 6.0 | 7.1 | 8.5 | 10.0 | 10.9 |
| (% Area ≦ 12.0) | | | | | | |

Reconstitution pH Results

The pH results for post-reconstitution in WFI were compared for formulations with a 25 mM citrate buffer system and formulations with a 10 mM citrate buffer system after storage for 0.5 months at 5° C. The results are shown in Table 2 and demonstrate that a 25 mM formulation provides an improved buffer capacity, thus maintaining a stable post-reconstitution pH.

TABLE 2

| Formulation | pH Start | pH End |
|---|---|---|
| 10 mM (PO14A) | 4.3 | 4.4 |
| 10 mM (PO14B) | 4.3 | 4.4 |
| 10 mM (PO14C) | 5.2 | 4.9 |
| 10 mM (PO14D) | 5.6 | 5.3 |
| 25 mM (PO15A) | 4.3 | 4.3 |
| 25 mM (PO15B) | 4.5 | 4.5 |
| 25 mM (PO15C) | 4.8 | 4.8 |
| 25 mM (PO15D) | 5.3 | 5.3 |

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. The disclosure of these publications is hereby incorporated by reference into this application to describe more fully the state of the art to which this invention pertains.

What is claimed is:

1. A freeze-dried formulation comprising a cephalosporin and derivatives thereof and a buffer system.

2. The formulation of claim 1, wherein the cephalosporin derivative is selected from a compound of Formula (I) or a pharmaceutically acceptable salt, stereoisomer, tautomer, crystalline, polymorph, amorphous, solvate, hydrate, ester, prodrug or metabolite thereof: 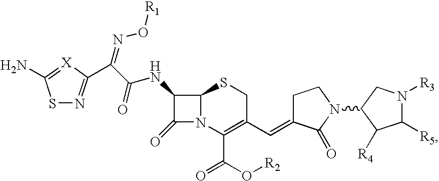
wherein
R₁ is hydrogen, C₁₋₆alkyl, optionally substituted by fluoro or C₃₋₆cycloalkyl;
R₂ is hydrogen or a group selected from —CH₂C(═CHR)—COOR, —CH₂OCOR, —CH(R)OCOR, —CH(R)OCOOR, —CH(OCOR)OCOR, —CH₂COCH₂OCOR and 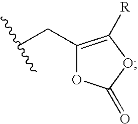
R₃ is hydrogen or a group selected from —CH₂C(═CH₂)—COOR, —COOCH₂C(═CHR)—COOR, —COOCH₂OCOR, —COOCH(R)OCOR, —COOCH(R)OCOOR, —COOCH(OCOR)OCOR, —COOCH₂COCH₂OCOR, and 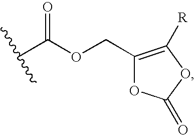
with the proviso that one of R₂ and R₃ is hydrogen and the other of R₂ and R₃ is different from hydrogen;
R is hydrogen or C₁₋₆alkyl;
R₄ is hydrogen or hydroxy;
R₅ is hydrogen or ω-hydroxyalkyl; and
X is CH or N.

3. The formulation of claim 2, wherein
R₁, R₂, R₄ and R₅ are all hydrogen;
R₃ is 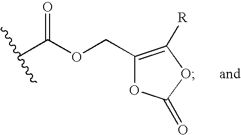
R is methyl.

4. The formulation of claim 1, wherein the compound of Formula (I) is (6R,7R)-7-[(Z)-2-(amino-[1,2,4]thiadiazol-3-yl)-2-hydroxyimino-acetylamino]-3-[(E)-(3′R, 5′R)-5′-hydroxymethyl-1′-(5-methyl-2-oxo-[1,3]dioxol-4-ylmethyloxycarbonyl-2-oxo-[1,3′]lbipyrrolidinyl-3-ylidenemethyl]-8-oxo-5-thia-1-aza-bicyclo[4.2.0]oct-2-ene-2-carboxylic acid or a pharmaceutically acceptable salt, stereoisomer, tautomer, crystalline, polymorph, amorphous, solvate, hydrate, ester, prodrug or metabolite thereof.

5. The formulation of claim 1, wherein the compound of Formula (I) is selected from a compound of Formula (Ia) or a pharmaceutically acceptable salt, stereoisomer, tautomer, crystalline, polymorph, amorphous, solvate, hydrate, ester, prodrug or metabolite thereof:

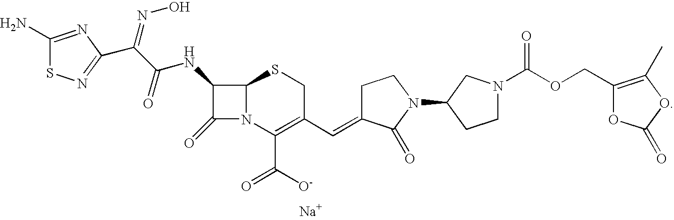

6. The formulation of claim 1, wherein the compound of Formula (I) is selected from a compound of Formula (Ib) or a pharmaceutically acceptable salt, stereoisomer, tautomer, crystalline, polymorph, amorphous, solvate, hydrate, ester, prodrug or metabolite thereof:

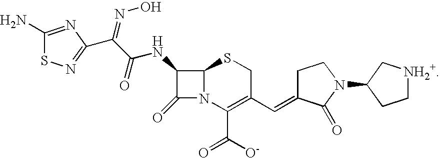

7. The formulation of claim 1, wherein the compound of Formula (I) is selected from a compound of Formula (Ic) or a pharmaceutically acceptable salt, stereoisomer, tautomer, crystalline, polymorph, amorphous, solvate, hydrate, ester, prodrug or metabolite thereof:

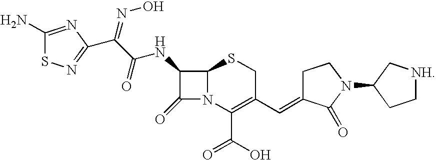

8. The formulation of claim 1, wherein the compound of Formula (I) is selected from a compound of Formula (Id) or a pharmaceutically acceptable salt, stereoisomer, tautomer, crystalline, polymorph, amorphous, solvate, hydrate, ester, prodrug or metabolite thereof:

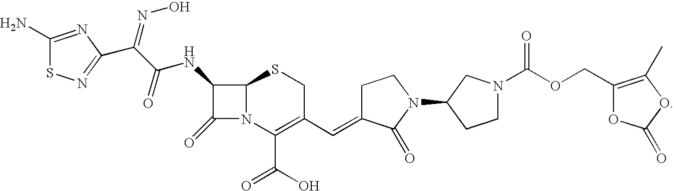

9. The formulation of claim 8, wherein the compound is a trihydrate hydrochloride salt.

10. The formulation of claim 2, wherein the freeze-dried formulation was prepared by bulk lyophilization.

11. The formulation of claim 5, wherein the freeze-dried formulation was prepared by bulk lyophilization.

12. The formulation of claim 8, wherein the freeze-dried formulation was prepared by bulk lyophilization.

13. The formulation of claim 9, wherein the freeze-dried formulation was prepared by bulk lyophilization.

14. The formulation of claim 1, wherein the formulation further comprises one or more additional optional ingredients selected from a bulking agent, a surfactant, a salt, a preservative, an antioxidant, a chelating agent or an optional cosolvent system.

15. The formulation of claim 1, wherein the buffer system comprises an acid, a base and a salt.

16. The formulation of claim 1, wherein the acid or base is mono-acidic, mono-basic, poly-basic or poly-acidic.

17. The formulation of claim 15, wherein the acid, base and salt is selected from acetate, acetic acid, arginine, ascorbate, ascorbic acid, bicarbonate, carbonate, carbonic acid, citrate, citric acid, glutamate, glutamic acid, glycine, histidine, hydrochloric acid, hydrogen carbonate, lactate, lactic acid, maleate, maleic acid, phosphate, phosphoric acid, potassium dihydrogen phosphate, potassium hydroxide, sodium dihydrogen phosphate, sodium hydroxide, succinate, succinic acid, tartrate, tartaric acid, tri(hydroxymethyl)aminomethane and combinations thereof.

18. The formulation of claim 17, wherein the acid, base and salt is selected from acetate, acetic acid, arginine, ascorbate, ascorbic acid, bicarbonate, citrate, citric acid, glutamate, glutamic acid, glycine, histidine, hydrochloric acid, lactate, lactic acid, phosphate, phosphoric acid, potassium dihydrogen phosphate, potassium hydroxide, sodium dihydrogen phosphate, succinate, succinic acid, tartrate, tartaric acid and combinations thereof.

19. The formulation of claim 17, wherein the acid, base and salt is selected from citrate, citric acid, glutamic acid, hydrochloric acid, phosphate, phosphoric acid and combinations thereof.

20. The formulation of claim 17, wherein the acid, base and salt is a combination of an acid, a base and a salt selected from potassium dihydrogen phosphate, phosphate/citrate, sodium dihydrogen phosphate or tartrate/citrate.

21. The formulation of claim 17, wherein the acid is selected from citric acid, glutamic acid, hydrochloric acid, phosphoric acid and combinations thereof; and, wherein the base is selected from potassium hydroxide or sodium hydroxide and combinations thereof.

22. The formulation of claim 17, wherein the acid is citric acid; and, wherein the base is sodium hydroxide.

23. The formulation of claim 15, wherein the buffer system is present in a concentration in a range of about 1 mM, or of about 10 mM, or of about 25 mM, or of from about 10 mM to about 25 mM, or of from about 10 mM to about 35 mM, or of from about 10 mM to about 40 mM, or of from about 10 mM to about 50 mM, or of from about 10 mM to about 100 mM, or of from about 25 mM to about 35 mM, or of from about 25 mM to about 40 mM, or of from about 25 mM to about 50 mM, or of from about 25 mM to about 100 mM, or of from about 25 mM to about 200 mM, or of from about 50 mM to about 200 mM.

24. The formulation of claim 23, wherein the buffer system is present in a concentration in a range of about 25 mM, or of from about 10 mM to about 50 mM, or of from about 25 mM to about 50 mM, or of from about 25 mM to about 200 mM, or of from about 50 mM to about 200 mM.

25. The formulation of claim 23, wherein the buffer system is present in a concentration in a range of about 25 mM, or of from about 10 mM to about 50 mM.

26. The formulation of claim 23, wherein the buffer system is present in a concentration in a range of about 25 mM.

27. The formulation of claim 14, wherein the bulking agent is selected from cellobiose, cyclodextrin, gelatin, gentiobiose, isomaltose, isosaccharose, isotrehalose, lactose, maltodextrins, maltose, melibiose, PVP, sorbose, sucralose, sucrose or trehalose or turanose.

28. The formulation of claim 27, wherein the bulking agent is present in a weight/weight ratio of bulking agent to the compound of claim 2 in a range of about 0:1, or of about 1:5, or of about 1:10, or of about 3:100, or of from about 1:10 to about 0:1, or of from about 1:10 to about 1:100, or of from about 1:100 to about 5:100, or of from about 1:200 to about 1:800, or of from about 1:250 to about 1:600, or of from about 1:100 to about 1:1500.

29. The formulation of claim 27, wherein the bulking agent is present in a weight/weight ratio of bulking agent to the compound of claim 2 of from about 1:100 to about 5:100, or of from about 1:200 to about 1:800, or of from about 1:250 to about 1:600, or of about 3:100.

30. The formulation of claim 27, wherein the bulking agent is present in a weight/weight ratio of bulking agent to the compound of claim 2 of about 3:100.

31. The formulation of claim 14, wherein the surfactant is selected from a phospholipid (such as lecithin), a polysorbate, a poloxamer (such as polyoxyethylene 20 sorbitan monooleate or polyoxyl 40 stearate), tyloxapol, a polyoxyethylene-polyoxypropylene copolymer (such as a Pluronic surfactant), a polyoxyethylene ester of 12-hydroxystearic acid (such as a Solutol surfactant), an ethoxylate of cholesterol (such as diacyl glycerol or dialkyl glycerol), a bile salt (such as sodium cholate or sodium deoxycholate), a sucrose ester (such as sucrose monolaurate or sucrose monooleate) or polyvinyl alcohol (PVA).

32. The formulation of claim 14, wherein the salt is selected from acetate, bicarbonate, chloride, glutamate, hydrochloride or sodium; an alkali metal sodium salt selected from ededate (tetrasodium EDTA), docusate (sodium 1,4-bis(2-ethylhexyl)sulphosuccinate), potassium or dipotassium carbonate; or an alkaline earth metal salt is selected from magnesium stearate or hydrates thereof.

33. The formulation of claim 14, wherein the preservative is selected from methyl and propyl para-hydroxybenzoate, benzethonium chloride, sodium mercurothiolate, phenylmercuric nitrate, benzyl alcohol, phenol or metacresol.

34. The formulation of claim 14, wherein the cosolvent system is selected from alcohols (such as methanol, ethanol, propanol, t-butanol), glycerin, polyethylene glycol, propylene glycol, vegetable oils and the like.

35. The formulation of claim 15, wherein the buffer system solubilizes the cephalosporin derivative prior to lyophilization.

36. The formulation of claim 15, wherein the buffer system modulates the bulk solution pH prior to lyophilization in a range of about pH 4.5 to about pH 5.6.

37. The formulation of claim 1, wherein the cephalosporin derivatives are selected from the compound of claim 5, the compound of claim 6, the compound of claim 7, the compound of claim 8, the compound of claim 9 and mixtures thereof.

38. The formulation of claim 37, wherein the cephalosporin derivatives are selected from the compound of claim 5, the compound of claim 6, the compound of claim 8, the compound of claim 9 and mixtures thereof.

39. The formulation of claim 1, wherein the formulation is reconstituted.

40. The formulation of claim 39, wherein the post-reconstitution formulation demonstrates stability of up to 24-30 hours at 25° C. or at least 48 hours at 5° C.

41. The formulation of claim 37, wherein the formulation is reconstituted.

42. The formulation of claim 41, wherein the post-reconstitution formulation demonstrates stability of up to 24-30 hours at 25° C. or at least 48 hours at 5° C.

43. The formulation of claim 38, wherein the formulation is reconstituted.

44. The formulation of claim 43, wherein the post-reconstitution formulation demonstrates stability of up to 24-30 hours at 25° C. or at least 48 hours at 5° C.

45. The formulation of claim 1, comprising the compound of claim 2 and the buffer system of claim 15.

46. The formulation of claim 45, wherein the formulation is reconstituted.

47. The formulation of claim 46, wherein the post-reconstitution formulation demonstrates stability of up to 24-30 hours at 25° C. or at least 48 hours at 5° C.

48. The formulation of claim 1, comprising the compound of claim 5 and the buffer system of claim 15.

49. The formulation of claim 48, wherein the formulation is reconstituted.

50. The formulation of claim 49, wherein the post-reconstitution formulation demonstrates stability of up to 24-30 hours at 25° C. or at least 48 hours at 5° C.

51. The formulation of claim 1, comprising the compound of claim 6 and the buffer system of claim 15.

52. The formulation of claim 51, wherein the formulation is reconstituted.

53. The formulation of claim 52, wherein the post-reconstitution formulation demonstrates stability of up to 24-30 hours at 25° C. or at least 48 hours at 5° C.

54. The formulation of claim 1, comprising the compound of claim 7 and the buffer system of claim 15.

55. The formulation of claim 54, wherein the formulation is reconstituted.

56. The formulation of claim 55, wherein the post-reconstitution formulation demonstrates stability of up to 24-30 hours at 25° C. or at least 48 hours at 5° C.

57. The formulation of claim 1, comprising the compound of claim 8 and the buffer system of claim 15.

58. The formulation of claim 57, wherein the formulation is reconstituted.

59. The formulation of claim 58, wherein the post-reconstitution formulation demonstrates stability of up to 24-30 hours at 25° C. or at least 48 hours at 5° C.

60. The formulation of claim 1, comprising the compound of claim 9 and the buffer system of claim 15.

61. The formulation of claim 60, wherein the formulation is reconstituted.

62. The formulation of claim 61, wherein the post-reconstitution formulation demonstrates stability of up to 24-30 hours at 25° C. or at least 48 hours at 5° C.

63. The formulation of claim 1, further comprising the compound of claim 5, one or more additional optional ingredients of claim 14, the buffer system of claim 15 and water for injection.

64. The formulation of claim 63, wherein the buffer system comprises citric acid and sodium hydroxide or potassium hydroxide.

65. The formulation of claim 63, wherein the buffer system comprises citric acid and sodium hydroxide.

66. The formulation of claim 63, wherein the buffer system comprises citric acid, potassium dihydrogen phosphate and sodium hydroxide or potassium hydroxide.

67. The formulation of claim 63, wherein the buffer system comprises citric acid, sodium dihydrogen phosphate, phosphoric acid and sodium hydroxide or potassium hydroxide.

68. The formulation of claim 63, wherein the buffer system comprises histidine, phosphoric acid and hydrochloric acid.

69. The formulation of claim 63, wherein the buffer system comprises glutamic acid and sodium hydroxide or potassium hydroxide.

70. The formulation of claim 63, wherein the buffer system comprises arginine and phosphoric acid.

71. The formulation of claim 63, wherein the buffer system comprises glycine, phosphoric acid and hydrochloric acid.

72. The formulation of claim 63, wherein the additional ingredient is sucrose; and, wherein the buffer system comprises citric acid and sodium hydroxide or potassium hydroxide.

73. The formulation of claim 63, wherein the additional ingredient is lactose; and, wherein the buffer system comprises citric acid and sodium hydroxide or potassium hydroxide.

74. The formulation of claim 63, wherein the additional ingredient is cyclodextrin; and, wherein the buffer system comprises citric acid and sodium hydroxide or potassium hydroxide.

75. The formulation of claim 63, wherein the additional ingredient is trehalose; and, wherein the buffer system comprises citric acid and sodium hydroxide or potassium hydroxide.

76. The formulation of claim 63, wherein the additional ingredient is sucralose; and, wherein the buffer system comprises citric acid and sodium hydroxide or potassium hydroxide.

77. The formulation of claim 63, wherein the additional ingredient is gelatin; and, wherein the buffer system comprises citric acid and sodium hydroxide or potassium hydroxide.

78. The formulation of claim 63, wherein the additional ingredient is anionic and non-ionic surfactants; and, wherein the buffer system comprises citric acid and sodium hydroxide or potassium hydroxide.

79. The formulation of claim 63, wherein the additional ingredient is anionic and non-ionic surfactants and a chelating agent; and, wherein the buffer system comprises citric acid and sodium hydroxide or potassium hydroxide.

80. A method for ameliorating, treating or preventing a chronic or acute disease mediated by anti-methicillin-resistant staphylococcus aureus, a Gram-positive bacteria or a Gram-negative bacteria in a subject in need thereof comprising administering to the subject an effective amount of the formulation of claim 39.

81. The method of claim 80, wherein the effective amount of the formulation of claim 39 is from about 250 mg to about 500 mg.

82. The method of claim 80, wherein the effective amount of the formulation of claim 41 is from about 250 mg to about 500 mg.

83. The method of claim 80, wherein the effective amount of the formulation of claim 43 is from about 250 mg to about 500 mg.

84. The method of claim 80, wherein the effective amount of the formulation of claim 46 is from about 250 mg to about 500 mg.

85. The method of claim 80, wherein the effective amount of the formulation of claim 49 is from about 250 mg to about 500 mg.

86. The method of claim 80, wherein the effective amount of the formulation of claim 52 is from about 250 mg to about 500 mg.

87. The method of claim 80, wherein the effective amount of the formulation of claim 55 is from about 250 mg to about 500 mg.

88. The method of claim 80, wherein the effective amount of the formulation of claim 58 is from about 250 mg to about 500 mg.

89. The method of claim 80, wherein the effective amount of the formulation of claim 61 is from about 250 mg to about 500 mg.

90. The formulation of claim 1, wherein the cephalosporin derivatives are present in a range of about 13.3 mg/ml, or of about 66.7 mg/ml, or of about 133.3 mg/ml, or of about 150.0 mg/ml, or of from about 13.3 mg/ml to about 199.5 mg/ml.

91. The formulation of claim 90, wherein the cephalosporin derivatives are present in a range of about 13.3 mg/ml, or of about 66.7 mg/ml, or of about 133.3 mg/ml.

92. Use of the formulation of claim 1 in the manufacture of a medicament for ameliorating, treating or preventing a chronic or acute disease mediated by anti-methicillin-resistant staphylococcus aureus, a Gram-positive bacteria or a Gram-negative bacteria.

93. The use of claim 92, wherein the cephalosporin derivatives are selected from the compound of claim 2, the compound of claim 5, the compound of claim 6, the compound of claim 7, the compound of claim 8, the compound of claim 9 and mixtures thereof.

94. The use of claim 93, wherein the cephalosporin derivatives are selected from the compound of claim 5, the compound of claim 6, the compound of claim 8, the compound of claim 9 and mixtures thereof.
